(19) United States
(12) Patent Application Publication
(10) Pub. No.: US 2024/0024664 A1
(43) Pub. Date: Jan. 25, 2024

(54) Methods of Treatment and Devices for Repair of Inflammatory, Neurotransmitter, Endocrine or Metabolic Issues

(71) Applicant: The Board of Trustees of the Leland Stanford Junior University, Stanford, CA (US)

(72) Inventor: Zara Marzban Patel, Woodside, CA (US)

(73) Assignee: The Board of Trustees of the Leland Stanford Junior University, Stanford, CA (US)

(21) Appl. No.: 18/044,970

(22) PCT Filed: Sep. 10, 2021
(86) PCT No.: PCT/US21/49945

§ 371 Date: Mar. 10, 2023

Related U.S. Application Data
(60) Provisional application No. 63/076,656, filed on Sep. 10, 2020.

(51) Int. Cl. A61N 1/05; A61B 5/05; A61B 1/00; A61N 1/36

(52) U.S. Cl. CPC A61N 1/0546; A61B 5/05; A61B 1/00082; A61N 1/36157; A61B 1/00124; A61B 1/0052

(57) ABSTRACT

Various methods and devices for treatment or assessment of structures or nerves accessible via the nasal cavity utilizing electrical stimulation or signal recording are provided. Devices can include one or more electrodes to provide electrical stimulation or signal recording. Electrodes can be disposed upon an inflatable balloon or a flat-surfaced tool or a precision tip.

### CROSS REFERENCE TO RELATED APPLICATIONS

This application claims priority to U.S. Provisional Application Ser. No. 63/076,656, entitled “Methods of Treatment and Devices for Repair of Neurotransmitter or Metabolic Issues,” filed Sep. 10, 2020, which is incorporated herein by reference in its entirety.

### TECHNICAL FIELD

The invention is generally directed to methods and devices involving treatments of olfactory loss, chronic rhinosinusitis, skull base, intracranial, and cranial nerve pathology and more specifically treatments of regenerating olfaction, improving cranial neuropathies, and treating skull base intracranial pathology via trans-nasal electrical stimulation.

### BACKGROUND

Olfaction is the sensing of odors. Humans detect odors through the olfactory system within the nose. The human olfactory system can detect thousands, and possibly millions, of odorant molecules, which are perceived to yield a smell. Lining a portion of the nasal cavity is the olfactory epithelium, a thin sheet of mucus-coated sensory tissue that contains the olfactory receptor cells (i.e., olfactory neurons), along with supporting cells and basal (stem) cells. Odorant molecules can reach the olfactory epithelium either via the nose, and/or from the mouth. Odorants dissolve into and pass through a layer of mucous overlying the olfactory epithelium and contact the olfactory receptor cells to induce the electrical signaling back to the olfactory bulb and brain allowing for the sensation of smell.

Olfaction is highly impactful on human wellbeing, social interactions, and quality of life. Loss of smelling function not only affects those important factors, but also can be a sign of dysfunction in higher cognitive ability and processing within the brain. Loss of smell leads to a greater than 3 times odds of mortality compared to normal smelling people, and this demonstrates both the importance of smell to our basic protective mechanisms as well as that smell dysfunction can be a sign of many other metabolic, endocrine, neurodegenerative or other systemic issues.

Chronic rhinosinusitis is a disease state of inflammation or infection involving the paranasal sinuses. The sinuses are lined with a mucosal layer made up of respiratory epithelium. This epithelium contains mucus producing glands as well as cells with tiny cilia (hairs) at their surface, which beat at a particular frequency to keep mucus moving at a normal rate and in the correct direction to clear and filter what is breathed into the nose and produced in the sinuses. Inflammation from multiple causes can lead to swelling of the lining, which in turn can cause mucus blockage which then becomes thickened and stagnant, and when it is not moving correctly, this can become a nidus for bacterial growth and infection.

Chronic rhinosinusitis is also highly impactful to quality of life and overall productivity, with yearly productivity costs greater than in those with chronic migraine, chronic asthma and diabetes, and health utility values similar to patients suffering from AIDS.

Cranial nerves control our sense of smell, our vision and ability to see clearly, the ability to move our eyes around and move them in the same direction at the same time in order to only see one clear image instead of multiple or double images, our ability to tear and cry and protect the cornea, our ability to move our facial muscles and therefore eat, smile, close and open our eyes and express emotion via facial expression, our ability to hear, and our ability to move our tongues and speak and swallow. Damage to or within these nerves can cause obvious deficits based on their functions outlined above.

The other ventral skull base structures of the brainstem, pons, pituitary and hypothalamus control multiple vital functions within the body involved with basic metabolic processes. The frontal and temporal lobes control multiple forms of higher-level functioning, including executive decision-making skills, personality and behavior control, memory, speech and hearing. All of these structures and the neurons within can become damaged and lead to changes in or loss of those functions.

### SUMMARY OF THE DISCLOSURE

Various embodiments are directed towards devices and methods for electrical stimulation to and/or electrical signal recording from biological activity in various tissues accessible via the nasal cavity and surrounding paranasal sinuses. In several embodiments, a device incorporates one or more electrodes at the distal end. In some embodiments, the one or more electrodes is an array. In many embodiments, the one or more electrodes are connected to an amplifier via an insulated track. In various embodiments, the one or more electrodes are disposed on an inflatable balloon or a flat surfaced tool. In several embodiments, the one or more distal electrodes are utilized in a procedure on a subject in which the one or more electrodes traverses through the nasal cavity to a tissue to be treated via electrical stimulation and/or assessed via electrical signal recording. In various embodiments, a tissue to be treated includes the olfactory epithelium, the respiratory epithelium within a sinus cavity, a cranial nerve, or an area of the brain in proximity to the skull base.

In an embodiment, an endoscopic device provides electrical stimulation or performing electrical recording. The device comprises a distal tool, a handle, and an amplifier system. The distal tool comprises endoscopic device for providing electrical stimulation or performing electrical recording and a connector. A portion of each electrode is exposed from the insulated track at the distal end of the distal tool. The connector of the distal tool connects with the handle such that the one or more electrodes is in conductive connection with the amplifier.

In an embodiment, a tool provides electrical stimulation or electrical signal recording. The tool comprises an inflatable balloon extending from a flexible arm at the distal end, a connector extending from the flexible arm at the proximal end, and one or more electrodes disposed on the inflatable balloon. Each electrode is insulated within a track but has an exposed portion on the inflatable balloon. Each insulated track runs along the flexible arm from the inflatable balloon to the connector. And each electrode is capable of being in conductive connection with an amplifier system via the connector.

In an embodiment, a tool provides electrical stimulation or electrical signal recording. The tool comprises a first flat flexible bayoneted surface extending from a first contoured arm at the distal end, a connector extending from the first contoured arm at the proximal end, one or more electrodes disposed on the flat flexible bayoneted surface. Each electrode is insulated within a track but has an exposed portion on the flat flexible bayoneted surface. Each insulated track runs along the first contoured arm from the first flat flexible bayoneted surface to the connector. And each electrode is capable of being in conductive connection with an amplifier system via the connector.

In a further embodiment, the tool further comprises a second flat flexible bayoneted surface at the distal end extending from a second contoured arm, and the connector extending from proximal end of the second contoured arm, forming a bifurcated tool. The second flat surface also comprises one or more distal electrodes disposed thereon.

In an embodiment, a tool provides electrical stimulation or electrical signal recording. The tool comprises a pinpoint electrode that incorporates the one or more electrodes, extending from the distal end of an insulated arm, and the pinpoint electrode having an exposed pinpoint-style distal tip. The tool further comprises a connector extending from the insulated arm at the proximal end. The pinpoint electrode is capable of being in conductive connection with an amplifier system via the connector.

In an embodiment, a method performs electrical stimulation to or electrical signal recording from tissue accessible via the nasal cavity, utilizing a distal tool with an inflatable balloon. The method comprises providing an endoscopic device. The endoscopic device comprises a distal tool, a handle, and an amplifier system. The distal tool comprises a flexible arm, an inflatable balloon extending from the distal end of the flexible arm, a connector extending from the proximal end of the flexible arm, and one or more electrodes disposed on the inflatable balloon and insulated within one or more tracks. A portion of each electrode is exposed from the insulated track at the inflatable balloon. The connector from the proximal end of the flexible arm connects with the handle such that the one or more electrodes is in conductive connection with the amplifier. The method further comprises advancing the inflatable balloon in a deflated state through the nasal cavity to a target site, inflating the balloon at the target site and positioning the one or more electrodes to be in proximity to tissue of the target site; and performing at least one of the following: recording electrical signal from the tissue of the target site using the one or more electrodes, or administering electrical stimulation to the tissue of the target site using the one or more electrodes.

In an embodiment, a method performs electrical stimulation to or electrical signal recording from tissue accessible via the nasal cavity, utilizing a distal tool with a flat bayoneted surface. The method comprises providing an endoscopic device. The endoscopic device comprises a distal tool, a handle, and an amplifier system. The distal tool comprises a first contoured arm, a first flat surface at the distal end of the first contoured arm, a connector extending from the proximal end of the contoured arm, and one or more electrodes disposed on the first flat surface and insulated within one or more tracks. A portion of each electrode is exposed from the insulated track at the first flat surface. The connector from the proximal end of the contoured arm connects with the handle such that the one or more electrodes is in conductive connection with the amplifier. The method further comprises advancing the first flat surface through the nasal cavity to a target site, positioning the one or more electrodes to be in proximity to tissue of the target site, and performing at least one of the following: recording electrical signal from the tissue of the target site using the one or more electrodes, or administering electrical stimulation to the tissue of the target site using the one or more electrodes.

In an embodiment, a method performs electrical stimulation to or electrical signal recording from tissue accessible via the nasal cavity, utilizing a pinpoint electrode distal tool. The method comprises providing an endoscopic device. The endoscopic device comprises a distal tool, a handle, and an amplifier system. The distal tool comprises a pinpoint electrode that incorporates the one or more electrodes, extending from an insulated arm and having an exposed pinpoint-style distal tip, and a connector extending from the proximal end of the insulated arm. The connector from the proximal end of the insulated arm connects with the handle such that the one or more electrodes is in conductive connection with the amplifier. The method further comprises advancing the pinpoint-style distal tip through the nasal cavity and traversing the skull base to a target site, positioning the one or more electrodes to be in proximity to tissue of the target site, and performing at least one of the following: recording electrical signal from the tissue of the target site using the one or more electrodes, or administering electrical stimulation to the tissue of the target site using the one or more electrodes.

### BRIEF DESCRIPTION OF THE DRAWINGS

The description and claims will be more fully understood with reference to the following figures and data graphs, which are presented as exemplary embodiments of the disclosure and should not be construed as a complete recitation of the scope of the various embodiments.

FIGS. 1A and 1B provide illustrations of exemplary endoscopic devices for trans-nasal electrical stimulation and electrical signal recording utilizing an inflatable balloon in accordance with various embodiments. The balloon is in the deflated configuration in FIG. 1A and dilated configuration in FIG. 1B.

FIG. 1C provides an illustration of an exemplary endoscopic device for trans-nasal electrical stimulation and electrical signal recording utilizing an inflatable balloon extended from a flexible, malleable, arm in accordance with various embodiments.

FIGS. 2A and 2B provide illustrations of exemplary tools with electrodes disposed on an inflatable balloon for trans-nasal electrical stimulation and electrical signal recording in accordance with various embodiments.

FIGS. 2C and 2D provide illustrations of exemplary tools with electrodes disposed on a bayonetted flat yet flexible surface for trans-nasal electrical stimulation and electrical signal recording in accordance with various embodiments.

FIGS. 3A to 3D provide illustrations of exemplary tools of precision pinpoint electrodes for trans-nasal electrical stimulation and electrical signal recording in accordance with various embodiments.

FIGS. 4A and 4B provide illustrations of exemplary methods to perform electrical stimulation to and electrical signal recording from the olfactory epithelium utilizing electrodes disposed on an inflatable balloon in accordance with various embodiments. FIG. 4A is provided with an axial view and FIG. 4B is provided with a sagittal view.

FIGS. 5A and 5B provide illustrations of exemplary methods to perform electrical stimulation to and electrical signal recording from the olfactory epithelium utilizing electrodes disposed on a bifurcated bayonetted flat yet flexible surface in accordance with various embodiments. FIG. 5A is provided with an axial view and FIG. 5B is provided with a sagittal view.

FIGS. 6A, 6B and 6C provide illustrations of exemplary methods to perform electrical stimulation to and electrical signal recording from within the maxillary sinus cavity utilizing electrodes disposed on an inflatable balloon in accordance with various embodiments. FIG. 6A is provided with an axial view, FIG. 6B is provided with a coronal view, and FIG. 6C is provided with a sagittal view.

FIGS. 7A, 7B and 7C provide illustrations of exemplary methods to perform electrical stimulation to and electrical signal recording from within the ethmoid sinus cavity utilizing electrodes disposed on an inflatable balloon in accordance with various embodiments. FIG. 7A is provided with an axial view, FIG. 7B is provided with a coronal view, and FIG. 7C is provided with a sagittal view.

FIGS. 8A, 8B and 8C provide illustrations of exemplary methods to perform electrical stimulation to and electrical signal recording from within the frontal sinus cavity utilizing electrodes disposed on an inflatable balloon in accordance with various embodiments. FIG. 8A is provided with an axial view, FIG. 8B is provided with a coronal view, and FIG. 8C is provided with a sagittal view.

FIGS. 9A, 9B and 9C provide illustrations of exemplary methods to perform electrical stimulation to and electrical signal recording from within the sphenoid sinus cavity utilizing electrodes disposed on an inflatable balloon in accordance with various embodiments. FIG. 9A is provided with an axial view, FIG. 9B is provided with a coronal view, and FIG. 9C is provided with a sagittal view.

FIGS. 10A and 10B provide illustrations of exemplary methods to perform electrical stimulation to and electrical signal recording from the cranial nerves and structures in proximity to the skull base utilizing precision pinpoint electrodes in accordance with various embodiments.

FIG. 11 provides a graph depicting the ability of Sprague-Dawley rats with injured olfactory epithelium to find food more rapidly after electrical stimulation treatment, generated in accordance with various embodiments. No electrical stimulation treatment was utilized as a control.

FIGS. 12A and 12B provides images of an olfactory epithelium of Sprague-Dawley rats with injured olfactory epithelium, generated in accordance with various embodiments. Electrical stimulation treatment resulted in more rapid normalization of neuroepithelial structure (FIG. 12A). No electrical stimulation treatment was utilized as a control (FIG. 12B).

### DETAILED DESCRIPTION

Turning now to the drawings and data, methods and devices for electrical stimulation and/or electrical signal recording from the olfactory system, nose and paranasal sinuses, cranial nerves and brain tissue in proximity to the skull base are provided in accordance with the various embodiments of the description. In several embodiments, an endoscopic device delivers electrical stimulation to and/or electrical signal recording from a tissue or an organ accessible via the nasal cavity. In many embodiments, an endoscopic device provides electrical stimulation to or electrical signal recording from an epithelium (e.g., olfactory epithelium), cavity (e.g., sinus cavity), a region within the skull base, a region within the brain, or a cranial nerve.

In several embodiments, an endoscopic electrical stimulation or signal recording device incorporates one or more electrodes for electrical stimulation and/or signal recording at the distal end of the device. In many embodiments, the one or more electrodes are in conductive connection with an amplifier via an insulated track. In various embodiments, the one or more electrodes can be disposed on the surface of a distal tool (e.g., balloon, a flat surfaced tool, a pinpoint electrode tool). In many embodiments, a pinpoint electrode tool incorporates one or more electrodes that extend from an insulated arm having an exposed pinpoint-style distal tip, which may be useful for precise electrical stimulation and/or signal recording. In several embodiments, an endoscopic device utilizes interchangeable distal tools (e.g., balloons with electrodes, flat flexible surface electrodes, and pinpoint tip electrodes) such that same device can be utilized to reach and provide electrical stimulation to and/or signal recording from various tissue architectures. For instance, various sized balloon shapes and sizes can be utilized to provide electrical stimulation to and/or signal recording from the various uniquely shaped sinus cavities, a flat-surfaced spatula can be utilized to provide electrical stimulation to and/or signal recording from relatively flat epithelial surfaces, and a pinpoint-styled electrode can be utilized to provide precise electrical stimulation to and/or signal recording from a cranial nerve or a particular region of the brain in proximity to the skull base.

Many embodiments are directed towards methods of endoscopic electrical stimulation and/or signal recording from via the nasal cavity. Accordingly, in several embodiments, an endoscopic device having one or more electrodes is traversed through the nasal cavity to reach an epithelium, a sinus cavity, or further traverses the skull base to reach an area of the brain in proximity to the skull base. To reach a site for electrical stimulation or signal recording, the distal portion of an endoscopic device can include a flexible and steerable arm attached to the balloon or spatula, such that the distal end can be flexed and steered around the various structures and turns within the nose and the paranasal sinus system.

Over 20 million Americans suffer from some level of olfactory loss. Over 80% of human ability to taste food and drink is dependent on olfaction, thus quality of life and sustained nutrition is greatly affected by loss of smell. Additionally, the olfactory system acts as a harbinger of many neurodegenerative diseases and mental disorders, factors largely in human social interaction and may soon be the basis for drug delivery to the brain to bypass the blood brain barrier. There are over 200 different etiologies for loss of olfaction, including inflammation, infection, trauma or degeneration. Various embodiments are directed towards electrically stimulating the olfactory epithelium and the end receptor neurons (collectively known as the olfactory nerve, or cranial nerve I) to incite or speed regeneration of the olfactory nerve after injury.

As noted above, chronic rhinosinusitis is also highly impactful to quality of life and overall productivity, with yearly productivity costs greater than in those with chronic migraine, chronic asthma and diabetes, and health utility values similar to patients suffering from AIDS. Various embodiments are directed towards trans-nasally electrically stimulating the respiratory epithelium and the cilia which transport mucus and keep the sinus lining healthy and functional to incite or speed regeneration of the cilia and respiratory epithelium after injury or inflammation.

Cranial nerves control our sense of smell, our vision and ability to see clearly, the ability to move our eyes around and move them in the same direction at the same time in order to only see one clear image instead of multiple or double images, our ability to tear and cry and protect the cornea, our ability to move our facial muscles and therefore eat, smile, close and open our eyes and express emotion via facial expression, our ability to hear, and our ability to move our tongues and speak and swallow. Damage to or within these nerves can cause obvious deficits based on their functions outlined above. Various embodiments are directed towards trans-nasally electrically stimulating the cranial nerves and their branches within the sinus and skull base region, to incite or speed regeneration of these nerves if damaged and not functional, or modulate or ablate these nerves if they are overactive or overly sensitized after injury or inflammation.

The other ventral skull base structures of the brainstem, pons, pituitary and hypothalamus control multiple vital functions within the body involved with basic metabolic processes. The frontal and temporal lobes control multiple forms of higher-level functioning, including executive decision-making skills, personality and behavior control, memory, speech and hearing. All of these structures and the neurons within can become damaged and lead to changes in or loss of those functions. Various embodiments are directed towards trans-nasally electrically stimulating these regions of the intracranial cavity and brain that are in proximity to the skull base region, to incite or speed regeneration of this neuronal tissue if damaged and not functional, or modulate or ablate the tissue if these regions are overactive or overly sensitized after injury or inflammation.

### Endoscopic Devices for Electrical Stimulation or Electrical Signal Recording

Several embodiments of the disclosure are directed to endoscopic devices for electrical stimulation and/or electrical signal recording. In many embodiments, an endoscopic device comprises one or more electrodes connected to an amplifier via an insulated track to provide electrical current or record electrical signals. In various embodiments, the one or more electrodes is disposed upon a surface of an inflatable balloon or of a flat surface tool (e.g., spatula or flat flexible electrode array). In some embodiments, the distal tip of one or more electrodes extends from an insulated arm, forming a pinpoint-style tip. In many embodiments, endoscopic devices comprising one or more electrodes are utilized to provide electrical stimulation to and/or electrical signal recording from various epitheliums, cavities, and tissues accessible via the nasal cavity, including (but not limited to) olfactory epithelium, the sinus cavities, cranial nerves and their associated branches and ganglia, and brain tissue in proximity with the skull base.

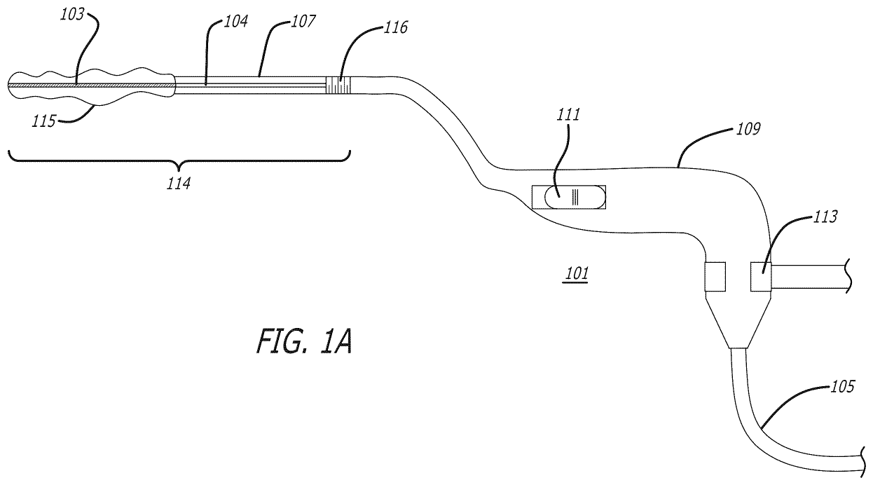

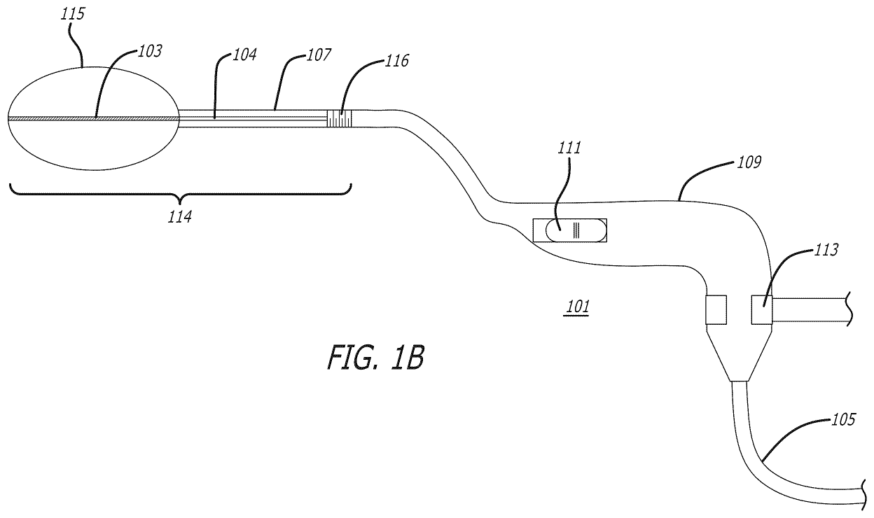

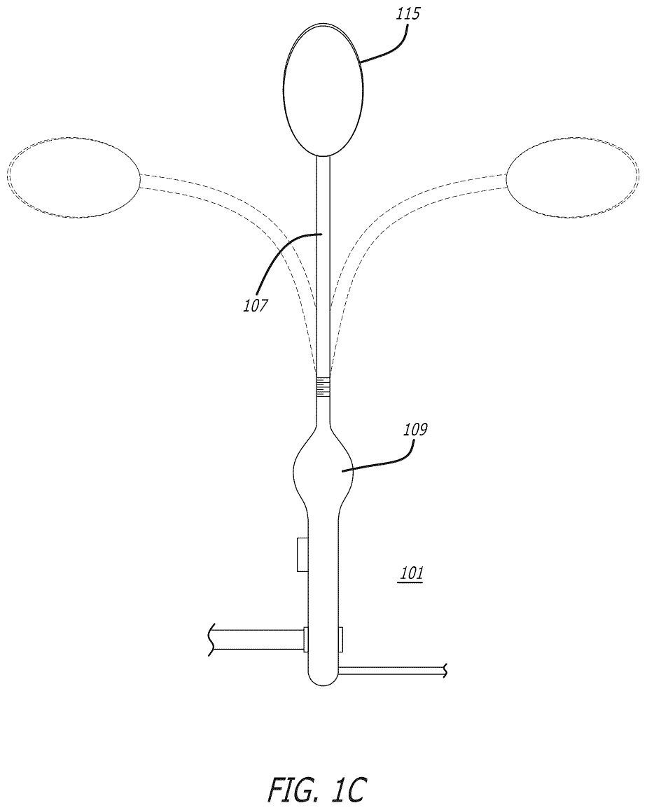

Provided in FIGS. 1A and 1B is an exemplary endoscopic device 101 for electrical stimulation and/or electrical signal recording. Endoscopic device 101 includes an electrode 103 at the distal end of the device, an insulated track 104, and a connection 105 to an amplifier system (not shown). Device 101 further includes a flexible arm 107 that connects the distal electrode 103 and insulated track 104 to the amplifier connection 105 via a handle 109. Device 101 can further include a power switch 111 for local control of power of the device and/or a stabilizing ring 113 for stabilizing control during usage or for storing the device.

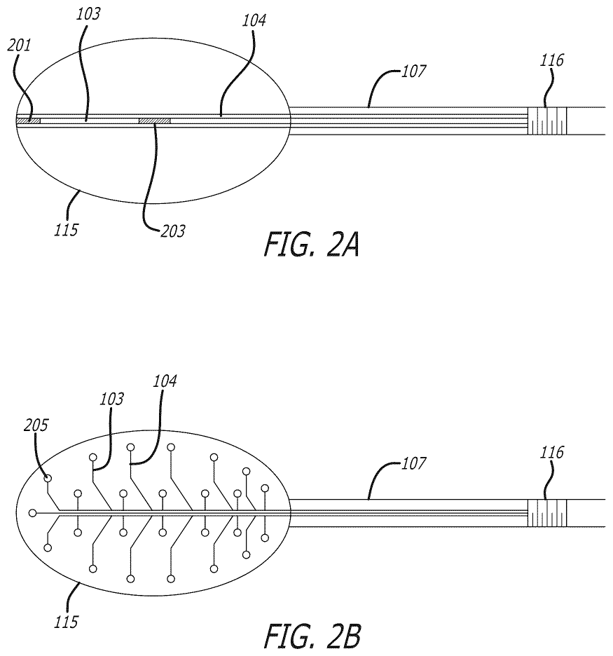

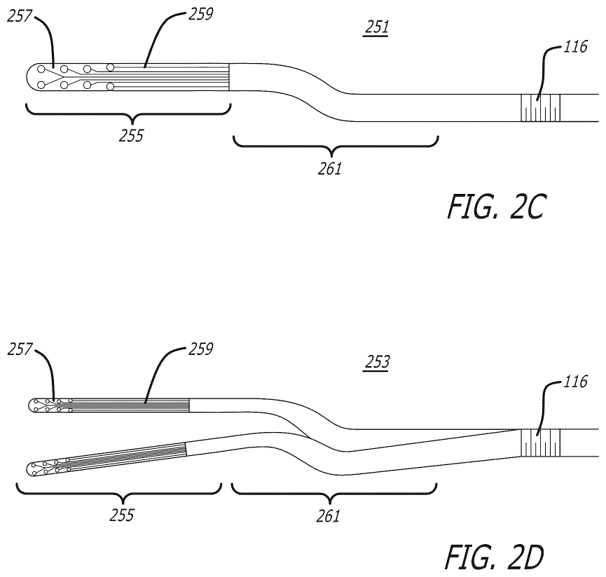

Although electrode 103 is depicted as a single electrode, several embodiments are directed to endoscopic devices having a plurality of electrodes. Each electrode (whether singular or within a plurality) can extend along a track and is in conductive connection with the amplifier system. Further, each can be embedded in a carrier material to insulate the electrode. Further, each electrode has an exposed portion to provide electrical stimulation and/or signal recording and is individually operable via the amplifier system. In many embodiments, an electrode terminal is exposed. As shown in FIGS. 1A and 1B, the portion of the electrode 103 exposed is the portion disposed on the balloon denoted by the shading, however, smaller or larger portions of the electrode can be exposed (see, e.g., FIGS. 2A and 2B). Any conductive material can be utilized as an electrode and any insulative material can be utilized to form a track. Conductive materials that can be utilized as an electrode include (but are not limited to), copper (Cu), graphite, titanium (Ti), brass, silver (Ag), gold (Au), platinum (Pt), titanium nitride (TiN), indium tin oxide (ITO), alloys thereof, and combinations thereof. Materials utilized to insulate electrodes (especially along the tracks) include (but are not limited to) glass, mineral oxides, silicon nitride (SiN), and various polymers. Further, any appropriate diameter of an electrode can be utilized, typically between 5 and 50 μm. In various embodiments, an electrode has a diameter of approximately 5 μm, 10 μm, 15 μm, 20 μm, 25 μm, 30 μm, μm, 40 μm, 45 μm, or 50 μm. The diameter size of an electrode can affect its impedance and spatial resolution. Larger electrodes will have decreased impedance but will also have decreased spatial resolution, as compared to electrodes of smaller size. Accordingly, electrode diameter can depend on the amount of stimulation to be provided or signal size to be recorded, and the spatial resolution desired.

As depicted in the example of FIGS. 1A and 1B, electrode 103 is disposed upon an inflatable balloon 115, which is depicted in a deflated configuration in FIG. 1A and dilated configuration in FIG. 1B. The balloon of the endoscopic device can be in connection with an air pump, fan, syringe, and/or vacuum system provide air and/or vacuum to inflate and deflate the balloon. In several embodiments, the balloon has a thin wall such that the balloon can conform to a cavity space as it is expanded. Any appropriate material can be utilized as the balloon wall, including (but not limited to) polyethylene terephthalate (PET), polytetrafluoroethylene (PTFE), nylon, and urethane. Further, various sizes of balloons can be utilized. In various embodiments, a dilated balloon has diameter between 5 and 40 mm, and a length between 10 and 50 mm. The size of the balloon utilized can vary and can be selected depending on the location of electrical stimulation and/or signal recording. For instance, the maxillary, the ethmoid, the frontal, and the sphenoid sinus cavities each have a unique volume and surface area distribution. Furthermore, each olfactory epithelium is a relatively flat surface of approximately 30 mm by 30 mm at the ceiling of the nasal canal.

A distal tool 114 of device 101 can be detachable. As shown in FIGS. 1A to 1C, the distal tool 114 can include the distal end of electrode 103, the inflatable balloon 115, and flexible arm 107. The distal tool 114 can connect to the handle via a connector 116. The connector 116 can utilize any reversible coupling mechanism to connect the distal tool 114 to the handle that allows for conductive connection of the electrode 103 to the amplifier system and airflow for inflating a balloon 115 (if applicable). Potential connectors include (but are not limited to) a threaded screw, a snap-on connector, hook with receiving groove, a flange, a twist lock, a ball and lock pin, or any capable combination of coupling mechanisms.

FIG. 1C depicts an example of a flexible arm 107 that connects the distal end of the electrode and the inflatable balloon 115 to the handle 109. Flexible arm 107 can be malleable in a way such that it can flex, bend, kink, and/or curve in any direction. Furthermore, inflatable balloon 115 and flexible arm 107 can be steerable utilizing principles of steerable catheters as known in the art.

A singular electrode 103 or a plurality of electrodes can be disposed on the inflatable balloon 115, as depicted in the examples provided in FIGS. 2A and 2B, respectively. For each electrode, in accordance with various embodiments, a portion of the electrode is exposed to provide electrical stimulation and/or signal recording and a portion of the electrode is insulated. When a single electrode (or a plurality of a few spaced out electrodes) is utilized, the portion of the electrode exposed can disposed upon any part of and can be of any length along the balloon. In some embodiments utilizing a single electrode (or a plurality of a few spaced out electrodes), the entire portion of the electrode disposed on the balloon is exposed (see, e.g., FIGS. 1A and 1B); and in some embodiments, only a portion of the electrode disposed on the balloon is exposed. In some embodiments utilizing a single electrode (or a plurality of a few spaced out electrodes), the portion of electrode exposed is located at the distal end 201 of the balloon; and in some embodiments, the portion of electrode exposed is located along a side 203 of the balloon. When a plurality of electrodes is utilized, the portion of exposed electrode an be positioned in such a way to prevent stimulation and/or signal interference with other electrodes, as is determined by the electrode spatial resolution. Inflatable balloon 115 and arm 107 can be connected to a handle of the device via a connector 116.

Several embodiments are directed to an array of electrodes disposed on the inflatable balloon (FIG. 2B). An array of electrodes can be provided in any pattern or shape, and the pattern or shape can be regular or irregular. In many embodiments utilizing an array of electrodes, each electrode terminus 205 is exposed and positioned in such a way to prevent electrical stimulation and/or electrical signal interference with another electrode termini, as is determined by the electrode spatial resolution. In several embodiments, each electrode has its own insulated track and is individually in connection with an amplifier system such that the amplifier system can provide electrical stimulation and/or signal recording from for each individual electrode. Accordingly, and in accordance with various embodiments, when electrical stimulation and/or signal recording is performed, a single electrode, a selection of a plurality of electrodes, or a plurality of all electrodes is utilized.

In accordance with many embodiments, devices with one or more electrodes disposed on an inflatable balloon are used for electrical stimulation and/or signal recording within a subject, especially within an area of the subject accessible via the nasal cavity. In various embodiments, devices with one or more electrodes disposed on an inflatable balloon are utilized for electrical stimulation to and/or signal recording from the olfactory epithelium and/or within the maxillary, the ethmoid, the frontal, and/or the sphenoid sinus cavities. In such embodiments, the balloon portion of the device traverses through the nasal cavity in a deflated state. Once the balloon portion reaches the target site for electrical stimulation and/or signal recording, the balloon can be inflated such that one or more electrodes are within proximity of an epithelium or sinus cavity wall and the one or more electrodes can be utilized to perform electrical stimulation and/or signal recording thereupon.

Several embodiments are directed to the use of a flat surface tool with one or more electrodes disposed thereupon. Provided in FIG. 2C is an example of a flat bayoneted tool 251 having an array of electrodes and in FIG. 2D is an example of a bifurcated flat bayoneted tool 253 having an array of electrodes on each arm for electrical stimulation and/or signal recording. As can be seen in these figures, the distal portion 255 of the tool contains one or two flat surfaces 257 (depending on whether a single arm or two bifurcated arms), upon which one or more electrodes 259 can be disposed. In some embodiments, the flat surfaces are flexible. Further, in a medial portion 261 of tool 251/253 are one or more curves to assist in navigating and/or locating the tool to the site for electrical stimulation and/or signal recording. In many embodiments, a flat surface tool is nonconductive or is coated with an insulative material to prevent electrode interference. Flat tool 251/253 can be connected to a handle of the device via a connector 116.

Many embodiments are directed to an array of electrodes disposed on the flat tool (FIGS. 2C and 2D). An array of electrodes can be provided in any pattern or shape, and the pattern or shape can be regular or irregular. In many embodiments utilizing an array of electrodes, each electrode terminus is exposed and positioned in such a way to prevent stimulation and/or signal interference with other electrode termini, as is determined by the electrode spatial resolution. In several embodiments, each electrode has its own track and insulation and is individually in connection with an amplifier such that the amplifier can provide electrical stimulation and/or signal recording from for each individual electrode. Accordingly, and in accordance with various embodiments, when electrical stimulation and/or signal recording is performed, a single electrode, a selection of a plurality of electrodes, or a plurality of all electrodes is utilized.

In accordance with several embodiments, devices with one or more electrodes disposed on a flat tool are used for electrical stimulation and/or signal recording within a subject, especially within an area of the subject accessible via the nasal cavity. In many embodiments, devices with one or more electrodes disposed on a flat tool are utilized for electrical stimulation to and/or signal recording from the olfactory epithelium. In such embodiments, the flat bayoneted portion of the device traverses through the nasal cavity to an area adjacent to the epithelium and the one or more electrodes can be utilized to perform electrical stimulation to and/or signal recording from thereupon. Curved portions can help navigate and/or locate the flat surface containing electrodes to reach the epithelium.

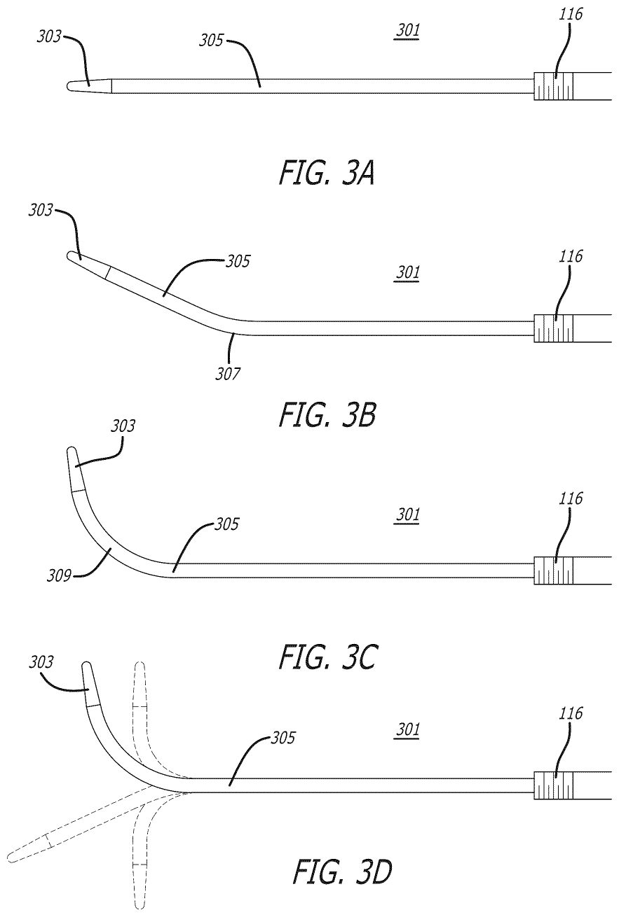

Several embodiments are directed to the use of a precision electrode, which is a single electrode (or a few bundled electrodes) that have an exposed distal tip extending from an insulated arm. Provided in FIGS. 3A to 3D are examples of precision electrodes 301 for electrical stimulation and/or signal recording. As can be seen in these figures, the distal tip 303 of the electrode is exposed and extends from an insulated arm 305. The insulated arm portion can be contoured in any way for navigation to reach a particular tissue area. For example, insulated arm portion can be straight (FIG. 3A), can include a bend or kink 307 (FIG. 3B), can include a curved portion 309 (FIG. 3C), or can be flexible such that it can bend or curve in any direction (FIG. 3D). Flexible arms can be steerable utilizing principles of steerable catheters as known in the art. Precision electrode 301 can be connected to a handle of the device via a connector 116.

In accordance with several embodiments, devices with a precision electrode are used for electrical stimulation and/or signal recording within a subject, especially within an area of the subject that is accessible via the nasal cavity. In many embodiments, devices with a precision electrode are utilized for electrical stimulation and/or signal recording from an area of the brain by traversing the skull base. In such embodiments, the precision electrode of the device traverses through the nasal cavity and through an incision in the skull base to an area of the brain in proximity to the skull base and the precision electrode can be utilized to perform electrical stimulation and/or signal recording thereupon. Bent, kinked, and/or curved portions can help navigate and/or locate the precision electrode to reach the area of stimulation.

In many embodiments, a distal tool of an endoscopic device is detachable and interchangeable such that various tools can be interchanged. Distal tools include (but are not limited to) balloons of various sizes, flexible flat surfaces (singular or bifurcated), and precision electrodes with various arm configurations. Accordingly, a single handle and amplifier can be utilized to perform various treatments and/or recordings of various tissues that are accessible via the nasal canal. It should be understood, however, that various embodiments are directed towards endoscopic devices with a dedicated (i.e., not interchangeable) distal tool, which may or may not be detachable. Accordingly, in various embodiments, a device can incorporate a dedicated balloon, flexible flat surface, or precision electrode.

In several embodiments, endoscopic devices with an electrode are connected to an amplifier system. In some embodiments, the amplifier system and device are further in connection with a computer, which can assist and/or automate the electrical stimulation or electrical recording to be performed. The amplifier system can provide the electrical conductance to the one or more electrodes to perform electrical stimulation. Likewise, the amplifier system can collect the electrical signals for analysis. A computer can provide further analysis of the electrical stimulation and/or electrical recording data generated.

In some embodiments, endoscopic devices are in connection with a visualization aid to help visualize the guidance of the distal portion of the device through the nasal canal and to the site of electrical stimulation and/or electrical recording. Radiographic imaging of the sinus and skull base region can be used for navigation and targeting within the sinuses, skull base or intracranial cavity, or simple endoscopic visualization can be used if navigation is not needed for more superficial structures within the nasal cavity, such as the olfactory epithelium.

### Treatment of Olfactory Loss, Chronic Rhinosinusitis, Cranial Neuropathy or Other Ventral Skull Base Pathology Utilizing Electrical Stimulation

Various embodiments are directed towards several methods for electrical stimulation and/or signal recording of the olfactory system, the cilia and cells within the sinonasal mucosal lining, the cranial nerves, and the brain regions in proximity to the ventral skull base. In some embodiments, electrical stimulation and/or signal recording is done in a procedure setting, where an electrode is introduced trans-nasally to stimulate the olfactory epithelium, the sinonasal lining, a cranial nerve or other structure within the intracranial cavity for a set period of time. In some embodiments, electrical stimulation is utilized to treat an injured or a dysfunctional tissue. In some embodiments, the length and width and material of the electrode is adjustable depending on how large of an electrical field is to be created. In some embodiments, this is done in an implant setting where the implant is placed submucosally (high within a mucosal pocket of the septum or superior turbinate where the olfactory fibers are), or a submucosal implant elsewhere in the sinuses, or an intracranial implant, and an external device could be used to turn it on and off. In various embodiments, if the portion of the olfactory system that is affected is the intracranial portion, or other cranial nerves or other intracranial skull base structures are to be treated, then Deep Brain Stimulation (DBS) could be used to stimulate those regions, accessed trans-nasally.

Many embodiments are directed towards assessing an individual for pathologies related to electrical signaling of the olfactory epithelium, cranial nerves, rhinosinusitis cavities, and/or other intracranial skull base pathology. Accordingly, an individual can be assessed as follows:
(i) advancing the distal tool with one or more electrodes through the nasal cavity to the target site of which electrical recording is to be performed
(ii) positioning the one or more electrodes in proximity to the tissue to be recorded
(iii) record electrical signals of olfactory epithelium, cranial nerves, paranasal sinus cavities and/or other regions of the ventral skull base

Several embodiments are directed towards treating an individual for olfactory loss, cranial nerve loss, chronic rhinosinusitis and other intracranial skull base pathology. Accordingly, an individual can be treated as follows:
(i) advancing the distal tool with one or more electrodes through the nasal cavity to the target site of which electrical stimulation is to be performed
(ii) positioning the one or more electrodes in proximity to the tissue to be stimulated
(iii) administer electrical stimulation to olfactory epithelium, to cranial nerves, to sinonasal mucosal surfaces, and/or other regions of the ventral skull base
(iv) repeat administration of electrical stimulation to olfactory epithelium, to cranial nerves, to sinonasal mucosal surfaces, and/or other regions of the ventral skull base as necessary

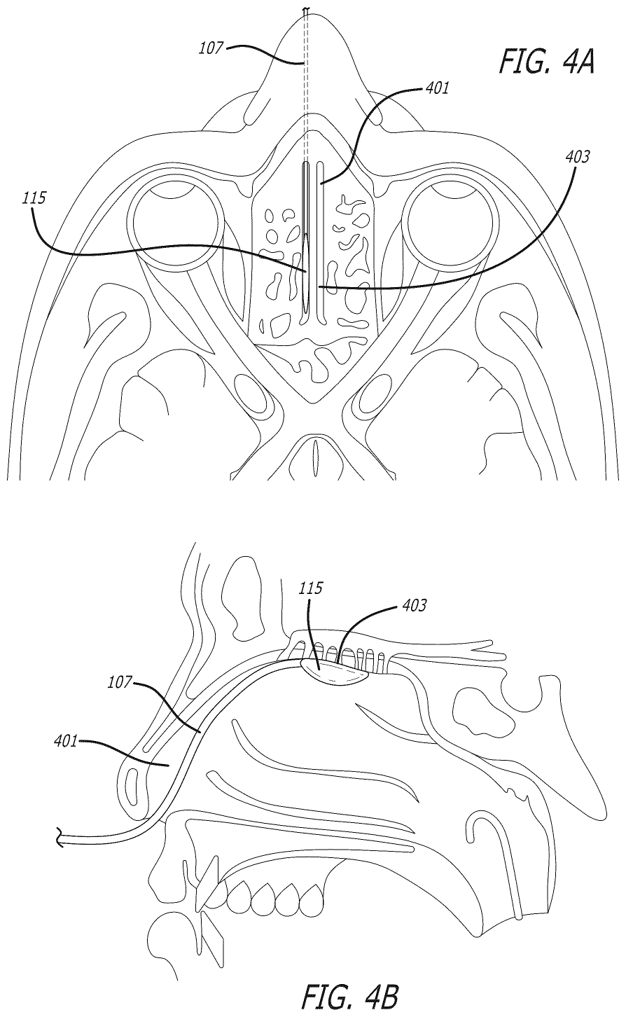

Provided in FIGS. 4A and 4B is an example of electrical stimulation and/or signal recording being performed on the olfactory epithelium with one or more electrodes disposed on an inflatable balloon. As can be seen in the figures, the distal portion of the endoscopic device comprising the inflatable balloon 115, one or more electrodes, and flexible arm 107 has traversed through a subject's nasal cavity 401 to the olfactory epithelium 403. The balloon can be advanced in a deflated state and inflated at the target site. To reach the olfactory epithelium 403, flexible arm 107 can bend and/or curve through the various canals such that inflatable balloon 115 is within proximity with the epithelium. Once in proximity, a selection of the one or more electrodes is utilized to perform electrical stimulation to and/or signal recording from the epithelium.

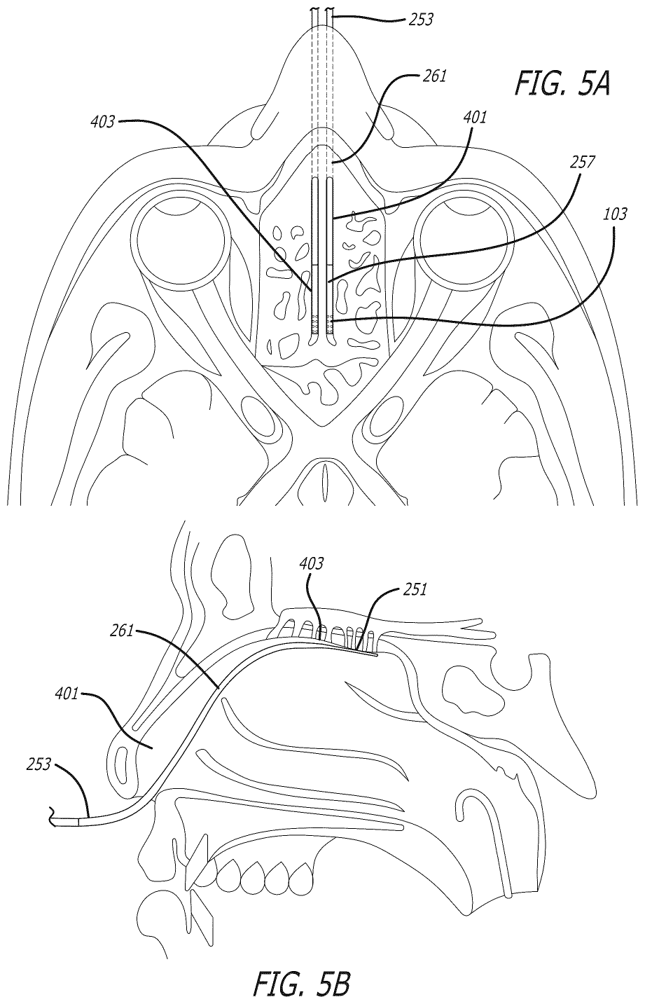

Provided in FIGS. 5A and 5B is an example of electrical stimulation and/or signal recording being performed on the olfactory epithelium with one or more electrodes disposed on a bifurcated flat tool. As can be seen in the figures, the distal portion of the endoscopic device comprising the bifurcated flat tool 253, one or more electrodes 103 on each arm of the spatula, and medial portion 261 has traversed through a subject's nasal cavity 401 to the olfactory epithelium 403. To reach the olfactory epithelium 403, the medial portion 261 can include one or more curves 261 to help navigate through the various canals such that flat surface 257 of the flat tool is within proximity with the epithelium. Once in proximity, a selection of the one or more electrodes is utilized to perform electrical stimulation to and/or signal recording from the epithelium.

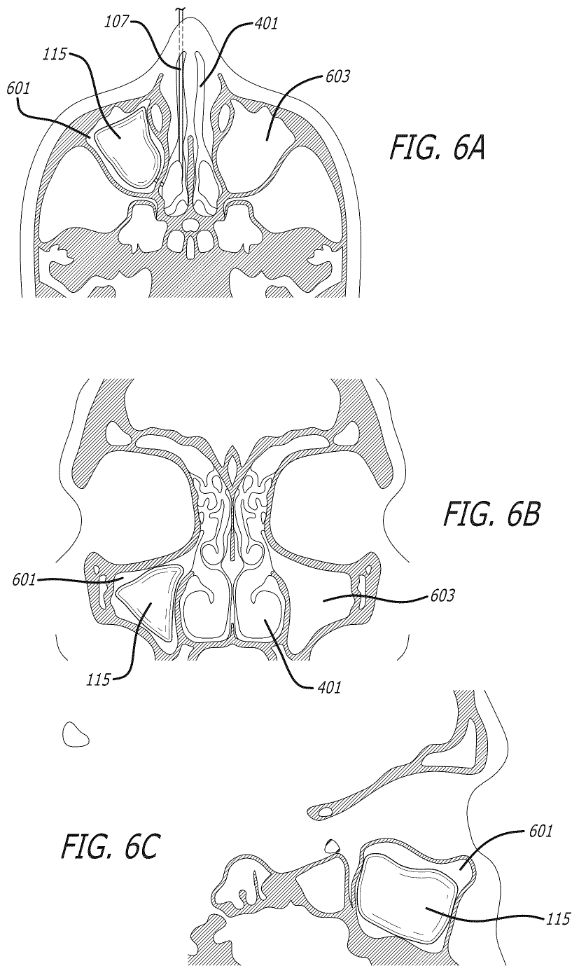

Provided in FIGS. 6A, 6B and 6C is an example of electrical stimulation and/or signal recording being performed on the maxillary sinus with one or more electrodes disposed on an inflatable balloon. As can be seen in the figures, the distal portion of the endoscopic device comprising the inflatable balloon 115, one or more electrodes, and flexible arm 107 has traversed through a subject's nasal canal 401 to the left maxillary sinus cavity 601. The balloon can be advanced in a deflated state and inflated at the target site. To reach the maxillary sinus cavity 601, flexible arm 107 can bend and/or curve through the various canals such that inflatable balloon 115 is within proximity with the cavity submucosal lining. Once in proximity, a selection of the one or more electrodes is utilized to perform electrical stimulation to and/or signal recording from the submucosal lining. It is to be understood, the right maxillary sinus cavity 603 can be treated and/or assessed in a similar fashion.

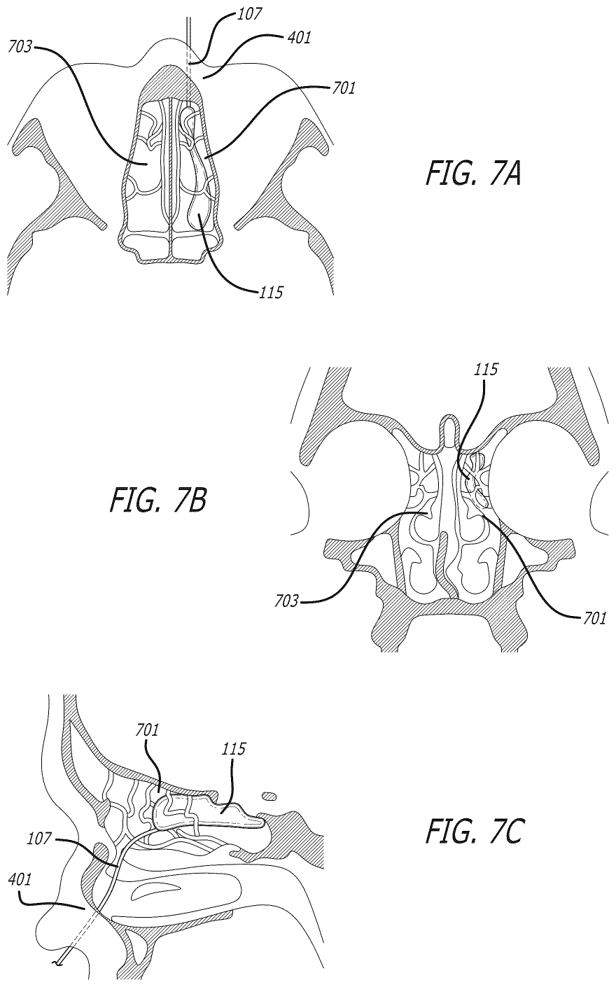

Provided in FIGS. 7A, 7B and 7C is an example of electrical stimulation and/or signal recording being performed on the ethmoid sinus with one or more electrodes disposed on an inflatable balloon. As can be seen in the figures, the distal portion of the endoscopic device comprising the inflatable balloon 115, one or more electrodes, and flexible arm 107 has traversed through a subject's nasal cavity 401 to the right ethmoid sinus cavity 701. The balloon can be advanced in a deflated state and inflated at the target site. To reach the ethmoid sinus cavity 701, flexible arm 107 can bend and/or curve through the various canals such that inflatable balloon 115 is within proximity with the cavity submucosal lining. Once in proximity, a selection of the one or more electrodes is utilized to perform electrical stimulation to and/or signal recording from the submucosal lining. It is to be understood, the left ethmoid sinus cavity 703 can be treated and/or assessed in a similar fashion.

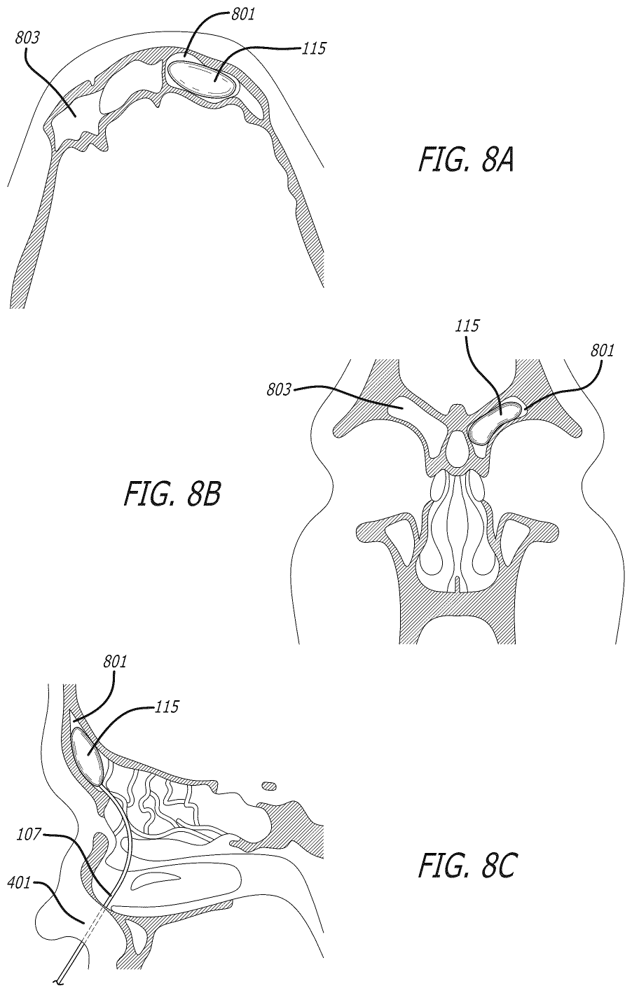

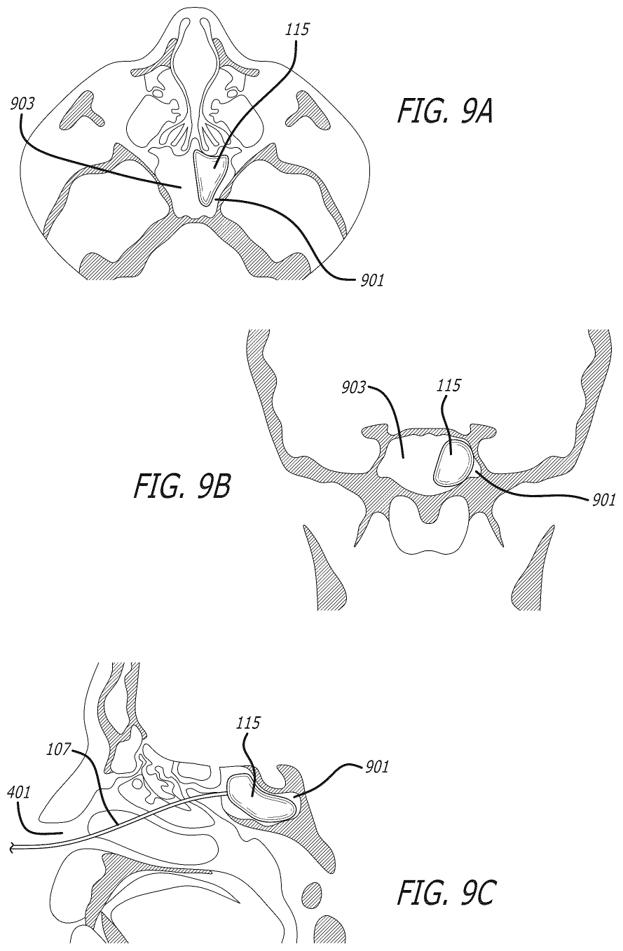

Provided in FIGS. 8A, 8B and 8C is an example of electrical stimulation and/or signal recording being performed on the frontal sinus with one or more electrodes disposed on an inflatable balloon. As can be seen in the figures, the distal portion of the endoscopic device comprising the inflatable balloon 115, one or more electrodes, and flexible arm 107 has traversed through a subject's nasal cavity 401 to the right frontal sinus cavity 801. The balloon can be advanced in a deflated state and inflated at the target site. To reach the frontal sinus cavity 801, flexible arm 107 can bend and/or curve through the various canals such that inflatable balloon 115 is within proximity with the cavity submucosal lining. Once in proximity, a selection of the one or more electrodes is utilized to perform electrical stimulation to and/or signal recording from the submucosal lining. It is to be understood, the left frontal sinus cavity 803 can be treated and/or assessed in a similar fashion.

Provided in FIGS. 9A, 9B and 9C is an example of electrical stimulation and/or signal recording being performed on the sphenoid sinus with one or more electrodes disposed on an inflatable balloon. As can be seen in the figures, the distal portion of the endoscopic device comprising the inflatable balloon 115, one or more electrodes, and flexible arm 107 has traversed through a subject's nasal canal 401 to the right sphenoid sinus cavity 901. The balloon can be advanced in a deflated state and inflated at the target site. To reach the sphenoid sinus cavity 901, flexible arm 107 can bend and/or curve through the various canals such that inflatable balloon 115 is within proximity with the cavity submucosal lining. Once in proximity, a selection of the one or more electrodes is utilized to perform electrical stimulation to and/or signal recording from the submucosal lining. It is to be understood, the left sphenoid sinus cavity 903 can be treated and/or assessed in a similar fashion.

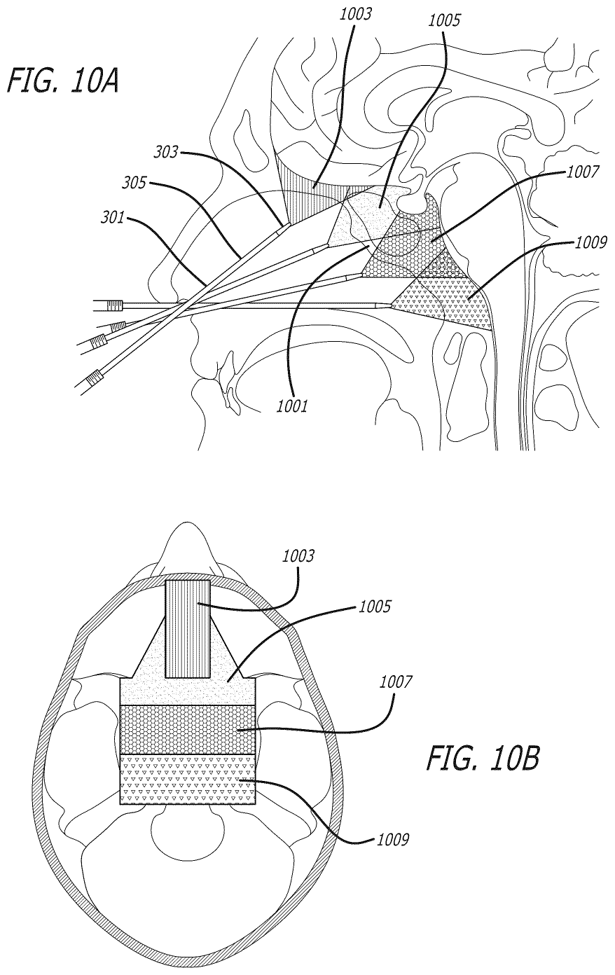

Provided in FIGS. 10A and 10B are various exemplary routes for electrical stimulation and/or signal recording of the various brain regions in proximity to the ventral skull base. As can be seen in FIG. 10A, the distal portion of the endoscopic device comprising the precision pinpoint-style electrode 301, it's distal tip 303, and the proximal portion of insulated track 305 can traverse through the nasal canal and an incision in the skull base to reach various brain regions. Depending on the region to be stimulated, the precision pinpoint-style electrode 301 can reach various structures in proximity to the skull base 1001. In various embodiments, the precision pinpoint-style electrode 301 is used to provide electrical stimulation to or record signal from any structure within or in proximity to the anterior skull base 1003, such as the olfactory receptor neurons, the olfactory bulb, the olfactory tract or other portions of the frontal lobes. In various embodiments, the precision pinpoint-style electrode 301 is used to provide electrical stimulation to or record signal from any structure within or in proximity to the suprasellar region 1005, such as the optic nerves, optic chiasm, pituitary stalk or infundibulum, the circle of Willis, the hypothalamus, the infundibular recesses of the third ventricle, or suprachiasmatic recesses of the third ventricle. In various embodiments, the precision pinpoint-style electrode 301 is used to provide electrical stimulation to or record signal from any structure within or in proximity to the sellar/parasellar region 1007, such as the pituitary gland, cavernous sinus, carotid arteries and associated sympathetic and parasympathetic nerve structures, cranial nerve 3, cranial nerve 4, cranial nerve 5, cranial nerve 6, nerves and ganglia associated with various cranial nerves, such as the vidian, the superficial branches of the trigeminal system that transmit sensation from the face and sinus region, the sphenopalatine ganglion, and other structures within the pterygopalatine fossa. In various embodiments, the precision pinpoint-style electrode 301 is used to provide electrical stimulation to or record signal from any structure within or in proximity to the clival/midbrain/brainstem region 1009, such as the pons, brainstem, superior cervical spine, cranial nerve 7, cranial nerve 8, cranial nerve 9, cranial nerve 10 cranial nerve 11, petrous apex, carotid arteries and associated sympathetic and parasympathetic nerve structures, or the temporal lobes. Once the electrode is in proximity to the tissue or nerve to be treated and/or assessed, the electrode is utilized to perform electrical stimulation and/or signal recording from the local nerve cells.

Dosing and therapeutic regimens can be administered appropriate to the injury to be treated. In some embodiments, electrical stimulation is administered in a therapeutically effective amount as part of a course of treatment. As used in this context, to “treat” means to ameliorate at least one symptom of the disorder to be treated or to provide a beneficial physiological effect. For example, one such amelioration of a symptom could be improvement in olfactory sensation (e.g., identification of odorants upon testing).

A therapeutically effective amount can be an amount sufficient to prevent reduce, ameliorate or eliminate the symptoms of olfactory loss, other cranial nerve loss, chronic rhinosinusitis, or other ventral skull base pathology. In some embodiments, a therapeutically effective amount is an amount sufficient to increase olfactory sensation, increase acuity or function of other cranial nerves, or increase acuity or function of other intracranial structures.

### Exemplary Embodiments

Trans-Nasal Electrical Stimulation of Olfactory Neurons May Improve Regeneration after Damage

Background: Olfaction is highly impactful on our wellbeing, social interactions, and quality of life. Loss of function not only affects those important factors, but also can be a window into the higher cognitive ability and processing of the brain. Current therapeutic options fall far short of cure. Electrical stimulation has been utilized for multiple purposes in both the central and peripheral nervous systems to improve neuronal regeneration. The purpose of this study was to examine the effects of electrical stimulation on olfactory nerves after damage to the mammalian olfactory epithelium.

Methods: Sprague-Dawley rats were injected intraperitoneally with methimazole, known to be olfactotoxic in rodents, and randomized to receive or not receive electrical stimulation to the olfactory epithelium (OE). Behavioral olfactory testing via food finding assays were carried out at baseline, one week and one month. Rats in each group were sacrificed during this time period for histologic sectioning and labeled with olfactory marker protein (OMP) antibody for qualitative comparison.

Results: The OE of animals that underwent trans-nasal electrical stimulation had improved quality and density of olfactory receptor cells, compared to those that did not, as indicated by anti-OMP immunofluorescence. Furthermore, on two-way ANOVA, stimulated animals had a significantly decreased latency on food finding assays compared to unstimulated animals (p=0.03).

Conclusions: This data suggests that electrical stimulation of the OE speeds recovery from methimazole induced damage. This modality would help human patients suffering from hyposmia and anosmia by improving neuronal regeneration.

### Introduction

The olfactory nerve is unique among all other cranial nerves in that it has the inherent ability to regenerate, and does so continuously throughout an individual's lifetime. However, damage to the olfactory epithelium or other parts of the olfactory system can halt this regeneration and lead to permanent loss of smell. Unfortunately, in the adult human olfactory system, when this occurs, we have very few treatment options, and all with limited efficacy. Currently, olfactory training along with topical and systemic steroids are the most promising treatment modalities, but even these modalities combined help only approximately half of our patients.

The sense of olfaction is commonly undervalued. Serving as an evolutionary defense mechanism, it allows avoidance smoke or other noxious fumes, as well as avoiding rotten food or drink. Additionally, the sense of smell plays a large role in how humans choose partners and lifelong mates and allows one to pick up on and respond to unspoken social cues. Due to the significant impact olfaction has on our ability to taste the flavor of food, both social interaction that commonly occurs over meals and drinks as well as satiation feedback with eating are negatively impaired by dysfunction, commonly leading to social isolation, depression, and swings to either extreme in body mass index (BMI). These factors, along with the multiple neurodegenerative and transmitter disease states for which olfactory loss is an early harbinger, lead to mortality rates over three times higher than those of normosmic people.

Electrical stimulation has been used in a variety of ways within the nervous system, ranging from deep brain stimulation to control motor function in Parkinson's patients, to decreasing pain via stimulation of peripheral sensory neurons, to direct translation of sight and sound via electrical signals. Specifically, in the context of nerve regeneration, electrical stimulation has been used in traumatic brain injury as well as both motor and sensory peripheral nerve injuries. The aim of the study was to examine if electrical stimulation would have a beneficial effect on olfactory nerve regeneration after injury.

### Methods

Sprague-Dawley rats were first given a behavioral food finding test as a measure of olfaction, using a common buried food pellet rodent protocol, to establish each animal's own baseline. Pellets were buried in standardized depths and rotating locations within 10 cm of bedding in otherwise clean cages. After a 24-hour period of food restriction, rats underwent three sequential timed food finding tests, with a cut-off time of 15 minutes. All rats then underwent chemical injury to their olfactory system via intraperitoneal administration of methimazole (300 mg/kg), an accepted methodology of rapid isolated disruption of the olfactory epithelium (Day 0). One day after injection, animals were randomized to receive electrical stimulation to the olfactory epithelium or not. This was carried out via placement of a thin platinum wire electrode inserted via the rat nostril into the pre-cribriform space intranasally, where the confluence of fibers from the olfactory epithelium coalesce and join together before passing through to the olfactory bulb within the intracranial cavity. Once positioned, an electrical stimulus was produced using a constant current pulse stimulator. A continuous train of 20 Hz square biphasic pulses of positive and negative 3V, 0.1 ms was delivered over one hour to the olfactory epithelium, a protocol based on prior studies using neurostimulation in sensory nerves.

Rats then again underwent food restriction at 6 days post-injury, and on day 7 were given the behavioral food finding test, again with 3 sequential tests per animal. Half these animals were then sacrificed, perfused, and underwent harvesting of the olfactory epithelium and staining with olfactory marker protein (OMP) to evaluate the neuronal structure and density within the olfactory epithelium (OE). This same protocol was repeated for the remaining animals at 30 days post-injury, with food restriction followed by food finding test, sacrifice, and harvesting of the olfactory epithelium.

### Results

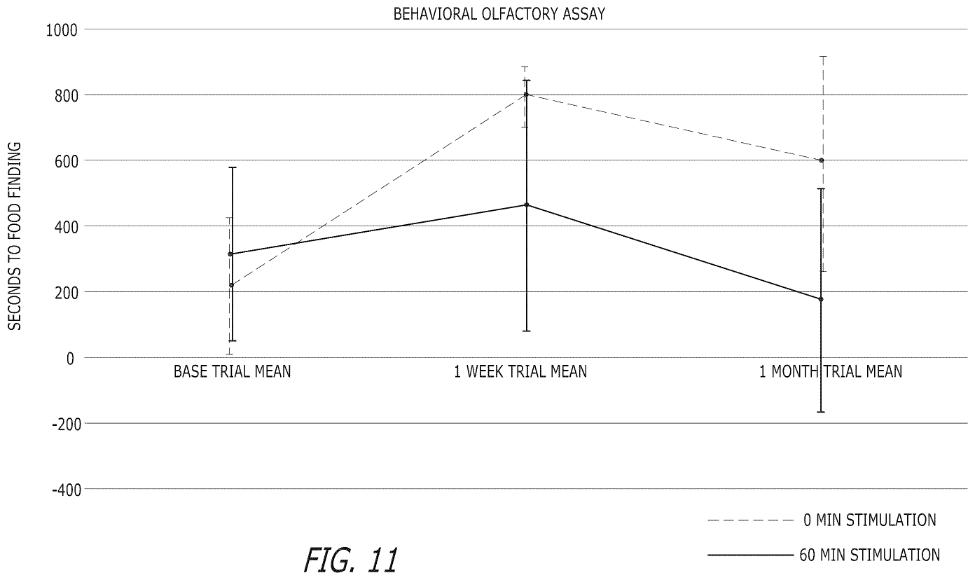

Baseline mean average time to food finding was slightly faster in the animal group that did not receive electrical stimulation at 221.67 seconds versus 319.11 seconds in the stimulation group, however while both groups had increased food finding times at one week, the group receiving stimulation had less of a change off their baseline. At one month, the electrical stimulation group had improved to a mean actually better than their baseline at 178.00 seconds, whereas the control group remained far off their baseline at 599.83 seconds. (FIG. 11)

On two-way ANOVA analysis, to evaluate the significance of this quantitative measure, stimulated animals had a significantly decreased latency on food finding assays compared to unstimulated animals (p=0.003).

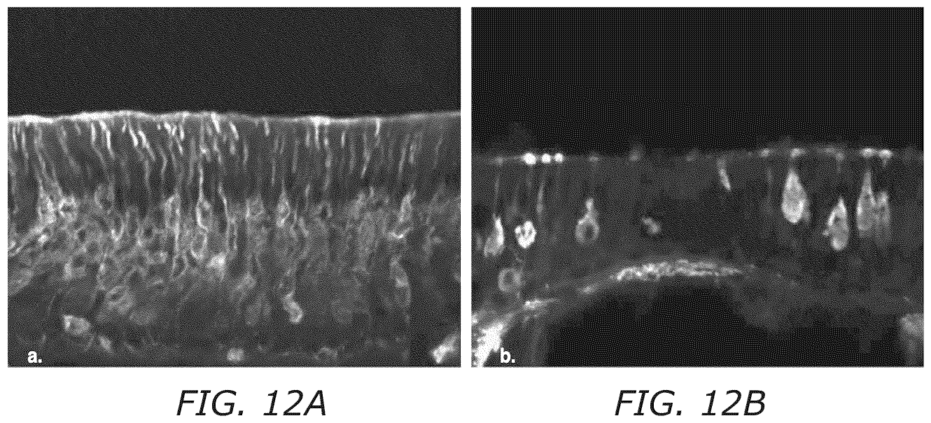

The qualitative measure of morphology and density of olfactory receptor neurons within the OE along the superior turbinates of these animals was observed after OMP staining and evaluation under confocal microscopy. While neither the animals who had received electrical stimulation, nor those that had not been stimulated, fully recovered back to normal within the thirty-day time period, the OE of animals that underwent trans-nasal electrical stimulation had improved quality and density of olfactory receptor cells, compared to those that did not, as indicated by anti-OMP immunofluorescence (FIGS. 12A and 12B).

### Discussion

With nearly one in four Americans over the age of 40 suffering from some level of alteration of smell (>20% of the population), and a current paucity of treatment options, any innovation in this field could be transformative in these patients' lives.

When olfactory nerve injury occurs, although regeneration is inherent to the system, as the Schwann-like ensheathing cells age or after a particularly inflammatory or damaging insult to the neurons, the expression of regenerative-associated genes declines over time and regeneration begins to fail. Although in rodents, olfactory ability eventually will progress back to normal, unfortunately in humans this does not always occur. Pioneering work in electrical stimulation has been shown to promote axonal regeneration, based on increased expression of regenerative associated genes such as brain derived neurotropic factor (BDNF), leading to increased intracellular cyclic adenosine monophosphate (cAMP) and thereby a sustained increase in expression of tubulin, actin and growth-associated protein (GAP-43). Although much of that work has been done after complete physical nerve transection, recent literature suggests the benefits of electrical stimulation for non-transection nerve injury as well.

The findings in this study demonstrate that electrical stimulation at this specific frequency allows for promotion of neuronal recovery while remaining low enough to prevent neuronal damage.

### CONCLUSION

This data suggests that electrical stimulation of the OE speeds recovery from methimazole induced damage and would help human patients suffering from hyposmia and anosmia by improving neuronal regeneration.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1. An endoscopic device for providing electrical stimulation or performing electrical recording, comprising:
a distal tool comprising:
one or more electrodes insulated within one or more tracks, wherein a portion of each electrode is exposed from the insulated track at a distal end of the distal tool; and
a connector;
a handle; and
an amplifier system;
wherein the connector of the distal tool connects with the handle such that the one or more electrodes is in conductive connection with the amplifier.

2. The device of claim 1, wherein the distal tool further comprises an inflatable balloon at the distal end of the distal tool and an arm extending from the inflatable balloon to the connector, and wherein the one or more distal electrodes are disposed on the inflatable balloon.

3. The device of claim 1, wherein the one or more electrodes is a plurality of electrodes arranged in an array, each electrode of the array having its own insulated track such that each electrode is individually in conductive connection with the amplifier system.

4. The device of claim 3, wherein each electrode of the array is individually operable via the amplifier system.

5. The device of claim 3, wherein each electrode has a terminus that is exposed.

6. The device of claim 5, wherein each electrode terminus is positioned in such a way to prevent electrical interference with another electrode terminus.

7. The device of claim 2, wherein the balloon is sized for an olfactory epithelium or a sinus cavity accessible via the nasal cavity.

8. (canceled)

9. (canceled)

10. The device of claim 2, wherein the arm is flexible and steerable.

11. (canceled)

12. The device of claim 1, wherein the distal tool further comprises a first flat surface at the distal end of the distal tool and a first arm extending from the flat surface to the connector, and wherein the one or more distal electrodes are disposed on the first flat surface.

13.-16. (canceled)

17. The device of claim 12, wherein the flat surface is sized for an olfactory epithelium accessible via the nasal cavity.

18. (canceled)

19. The device of claim 12, wherein the arm includes a curve in the medial portion.

20. The device of claim 12, wherein
the distal tool further comprises a second flat surface at the distal end of the distal tool and a second arm extending from the flat surface to the connector, forming a bifurcated tool, and wherein the second flat surface also comprises one or more distal electrodes disposed thereon.

21. The device of claim 1, wherein the distal tool comprises a pinpoint electrode that incorporates the one or more electrodes, extending from an insulated arm and having an exposed pinpoint-style distal tip.

22. The device of claim 21, wherein the insulated arm includes a bend, a curve, or a kink.

23. The device of claim 21, wherein the insulated arm is flexible.

24. The device of claim 23, wherein the insulated arm is steerable.

25. The device of claim 1, wherein the distal tool is detachable.

26. The device of claim 1 further comprising a set of two or more interchangeable distal tools, wherein each distal tool comprises:
one or more electrodes insulated within one or more tracks, wherein a portion of each electrode is exposed from the insulated track at the distal end of the distal tool; and
a connector.

27. The device of claim 26, wherein the set of two or more interchangeable distal tools includes a first distal tool and second distal tool;
wherein the first distal tool comprises one of the following:
an inflatable balloon at the distal end of the distal tool and an arm extending from the inflatable balloon to the connector, and wherein the one or more distal electrodes are disposed on the inflatable balloon;
a first flat surface at the distal end of the distal tool and a first arm extending from the flat surface to the connector, and wherein the one or more distal electrodes are disposed on the first flat surface; or
a pinpoint electrode that incorporates the one or more electrodes, extending from an insulated arm and having an exposed pinpoint-style distal tip; and
wherein the second distal tool comprises one of the following:
an inflatable balloon at the distal end of the distal tool and an arm extending from the inflatable balloon to the connector, and wherein the one or more distal electrodes are disposed on the inflatable balloon;
a first flat surface at the distal end of the distal tool and a first arm extending from the flat surface to the connector, and wherein the one or more distal electrodes are disposed on the first flat surface; or
a pinpoint electrode that incorporates the one or more electrodes, extending from an insulated arm and having an exposed pinpoint-style distal tip.

28. The device of claim 26, wherein the set of two or more interchangeable distal tools includes a first distal tool and second distal tool;
wherein the first distal tool comprises:
an inflatable balloon at the distal end of the distal tool and an arm extending from the inflatable balloon to the connector, and wherein the one or more distal electrodes are disposed on the inflatable balloon; and
wherein the second distal tool comprises:
an inflatable balloon at the distal end of the distal tool and an arm extending from the inflatable balloon to the connector, and wherein the one or more distal electrodes are disposed on the inflatable balloon;
wherein the size of the inflatable balloon of the first distal tool is different than the size of the inflatable balloon of the second distal tool.

29. The device of claim 26, wherein the set of two or more interchangeable distal tools includes a first distal tool and second distal tool;
wherein the first distal tool comprises:
a first flat surface at the distal end of the distal tool and a first arm extending from the flat surface to the connector, and wherein the one or more distal electrodes are disposed on the first flat surface; and
wherein the second distal tool comprises:
a first flat surface at the distal end of the distal tool and a first arm extending from the flat surface to the connector, and a second flat surface at the distal end of the distal tool and a second arm extending from the flat surface to the connector, forming a bifurcated tool, and wherein the first flat surface and the second flat surface each comprises one or more distal electrodes disposed thereon.

30. The device of claim 26, wherein the set of two or more interchangeable distal tools includes a first distal tool and second distal tool;
wherein the first distal tool comprises:
a pinpoint electrode that incorporates the one or more electrodes, extending from an insulated arm and having an exposed pinpoint-style distal tip; and
wherein the second distal tool comprises:
a pinpoint electrode that incorporates the one or more electrodes, extending from an insulated arm and having an exposed pinpoint-style distal tip;
wherein the contour of the insulated arm of the first distal tool is different than the contour of the insulated arm of the second distal tool.

31. A tool for providing electrical stimulation or electrical signal recording, comprising:
an inflatable balloon extending from a flexible arm at the distal end;
a connector extending from the flexible arm at the proximal end; and
one or more electrodes disposed on the inflatable balloon, wherein each electrode is insulated within a track but has an exposed portion on the inflatable balloon, wherein each insulated track runs along the flexible arm from the inflatable balloon to the connector, and wherein each electrode is capable of being in conductive connection with an amplifier system via the connector.

32. The tool of claim 31, wherein the one or more electrodes is a plurality of electrodes arranged in an array, each electrode of the array having its own insulated track such that each electrode is capable of being individually in conductive connection with the amplifier system.

33. The tool of claim 32, wherein each electrode has a terminus that is exposed.

34. The tool of claim 33, wherein each electrode terminus is positioned in such a way to prevent electrical interference with another electrode terminus.

35. The tool of claim 31, wherein the balloon is sized for an olfactory epithelium or a sinus cavity accessible via the nasal cavity.

36. (canceled)

37. (canceled)

38. The device of claim 31, wherein the flexible arm is steerable.

39.-50. (canceled)

51. A method of performing electrical stimulation to or electrical signal recording from tissue accessible via the nasal cavity, utilizing a distal tool with an inflatable balloon, comprising:
providing an endoscopic device comprising:
a distal tool comprising:
a flexible arm;
an inflatable balloon extending from the distal end of the flexible arm;
a connector extending from the proximal end of the flexible arm; and
one or more electrodes disposed on the inflatable balloon and insulated within one or more tracks, wherein a portion of each electrode is exposed from the insulated track at the inflatable balloon;
a handle; and
an amplifier system;
wherein the connector from the proximal end of the flexible arm connects with the handle such that the one or more electrodes is in conductive connection with the amplifier;
advancing the inflatable balloon in a deflated state through the nasal cavity to a target site;
inflating the balloon at the target site and positioning the one or more electrodes to be in proximity to tissue of the target site; and
performing at least one of the following:
recording electrical signal from the tissue of the target site using the one or more electrodes; or
administering electrical stimulation to the tissue of the target site using the one or more electrodes.

52. The method of claim 51, wherein the target site is an epithelium or a sinus cavity.

53. The method of claim 52, wherein the epithelium is an olfactory epithelium.

54. The method of claim 52, wherein the sinus cavity is a maxillary sinus, an ethmoid sinus, a frontal sinus, or a sphenoid sinus.

55. The method of claim 54, wherein the tissue of the target site is a sinonasal mucosal surface.

56. The method of claim 51, wherein the tissue of the target site is injured or dysfunctional.

57. The method of claim 51, wherein the one or more electrodes is a plurality of electrodes arranged in an array, each electrode of the array having its own insulated track such that each electrode is individually in conductive connection with the amplifier system.

58. The method of claim 57, wherein a subset of the plurality of electrodes is utilized for the performing of the recording electrical signal or the administering electrical stimulation.

59. The method of claim 57, wherein the entire array of electrodes is utilized for the performing of the recording electrical signal or the administering electrical stimulation.

60. The method of claim 51, wherein a visualization aid is utilized for the positioning of the one or more electrodes.

61.-82. (canceled)
